(19) United States
(12) Patent Application Publication
(10) Pub. No.: US 2010/0174075 A1
(43) Pub. Date: Jul. 8, 2010

(54) USE OF NITRIC OXIDE-RELEASING STATINS IN THE TREATMENT OF PULMONARY ARTERIAL HYPERTENSION

(75) Inventors: John Wharton, London (GB); Angela Monopoli, Milano (IT)

Correspondence Address: ARENT FOX LLP, 1050 CONNECTICUT AVENUE, N.W., SUITE 400, WASHINGTON, DC, 20036, US

(73) Assignee: NICOX S.A., SOPHIA ANTIPOLIS-VALBONNE (FR)

(21) Appl. No.: 12/599,240

(22) PCT Filed: May 12, 2008
(86) PCT No.: PCT/EP08/55788

§ 371 Date: Nov. 6, 2009

Related U.S. Application Data
(60) Provisional application No. 60/929,390, filed on Jun. 25, 2007.

(51) Int. Cl. C07D 239/42; C07D 209/10; C07D 207/34; C07C 69/34; C07D 213/02

(52) U.S. Cl. 544/297; 548/491; 548/537; 560/194; 546/335

(57) ABSTRACT

The present invention relates to the use of nitric oxide-releasing statins of formula (I) for the treatment of pulmonary arterial hypertension.

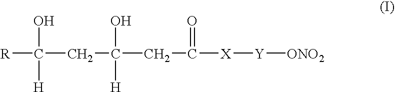

### FIELD OF THE INVENTION

The present invention relates to the use of nitric oxide (NO)-releasing statins for the treatment of pulmonary arterial hypertension.

Pulmonary arterial hypertension (PAH) is a progressive disease that is characterized by endothelial dysfunction, increased pulmonary vascular resistance and remodelling of distal pulmonary arteries, leading to right heart failure and premature death. Structural changes in the pulmonary vasculature are thought to be the consequence of an imbalance between proliferation and apoptosis of distal pulmonary artery smooth muscle cells (PASMCs). The mechanisms underlying the remodelling of pulmonary arteries in PAH are multi-factorial, and involve abnormal endothelin-1 (ET-1), serotonin, transforming growth factor (TGF-β1) and platelet-derived growth factor (PDGF) signalling and resistance to apoptosis (Runo J R. et al., The Lancet 2003, 361:1533-1544).

In addition, proteolytic enzymes such as elastase and the matrix metalloproteinases MMP-2 and MMP-9 are implicated in modulating the migration, proliferation and apoptosis of cells in the hypertensive pulmonary vessel wall (Frisdal E. et al., Eur Respir J 2001, 18:838-845; Lepetit H. et al., Eur Respir J 2005, 25:834-842).

It is well recognised that 3-hydroxy-3-methylglutaryl-coenzyme A (HMG-CoA) reductase inhibitors (statins) exhibit beneficial cardiovascular effects beyond cholesterol lowering such as anti-proliferative, anti-thrombotic, anti-inflammatory and anti-oxidant effects.

However, it is also known that statins exhibit adverse effects, such as for example gastrointestinal disturbances, hepatitis, pancreatitis, myopathy and rhabdomyolysis (Martindale, The complete drug reference, 33^{rd} edition, 969).

WO 2004/105754 discloses new nitroderivatives of statins showing an improved overall profile as compared to native statins both in terms of wider pharmacological activity and enhanced tolerability. In particular, the patent application describes statin nitroderivatives as compounds having anti-inflammatory, antithrombotic and antiplatelet activity, used for treating and/or preventing acute coronary syndromes, stroke, peripheral vascular diseases, such as peripheral ischemia, vascular complications in diabetic patients, atherosclerosis, neurodegenerative disorders, such as Alzheimer's and Parkinson's disease as well as autoimmune diseases such as multiple sclerosis.

It has now been surprisingly found that nitric oxide-releasing statins can be useful in the treatment of pulmonary arterial hypertension.

Object of the present invention is, therefore, the use in the treatment of pulmonary arterial hypertension of NO-releasing statins of general formula (I) or pharmaceutically acceptable salts or stereoisomers thereof

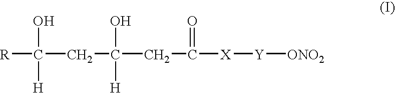

### R is

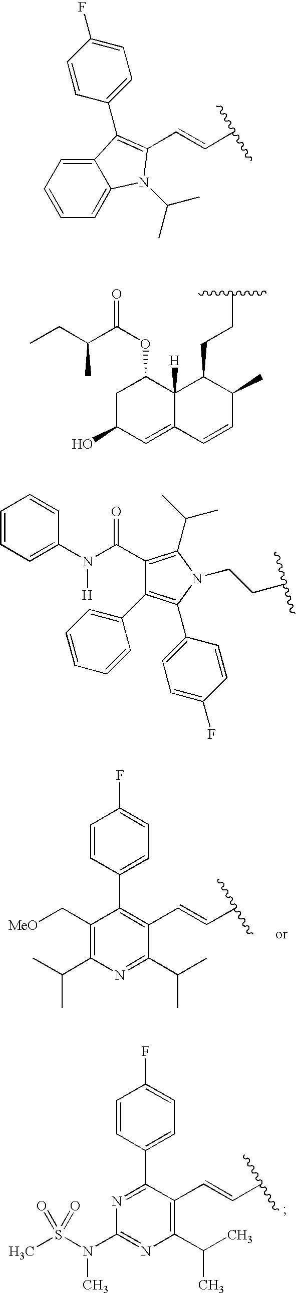

X is —O—, —S—, —NH— or —NHR′—, R′ being straight or branched alkyl with 1 to 10 carbon atoms, preferably CH₃;
Y is a bivalent radical having the following meaning:

a)
straight or branched C₁-C₂₀-alkylene, preferably having from 2 to 5 carbon atoms;
cycloalkylene with 5 to 7 carbon atoms into cycloalkylene ring, the ring being eventually substituted with side chains T, wherein T is straight or branched alkyl with from 1 to 10 carbon atoms, preferably CH₃;

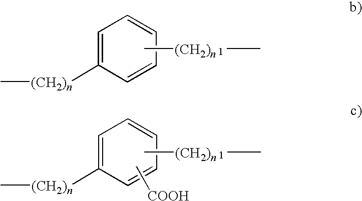

wherein n is an integer from 0 to 20, and n¹ is an integer from 1 to 20;

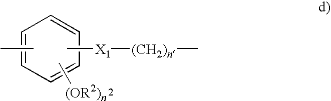

wherein:
n¹ is as defined above and n² is an integer from 0 to 2;

### X₁=—OCO— or —COO— and R² is H or CH₃;

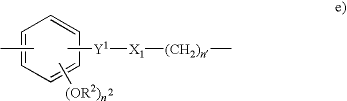

wherein:
n¹, n², R² and X₁ are as defined above;
Y¹ is —CH₂—CH₂— or —CH═CH—(CH₂)ₙ²—;

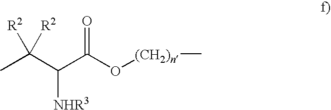

wherein:
n¹ and R² are as defined above, R³ is H or COCH₃; with the proviso that when Y is selected from the bivalent radicals mentioned under b)-f), the —ONO₂ group is linked to a —CH₂ group;

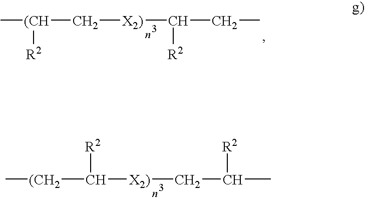

wherein X₂ is —O— or —S—, n³ is an integer from 1 to 6, preferably from 1 to 4, R² is as defined above;

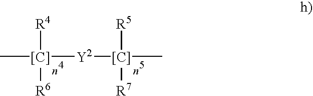

wherein:
n⁴ is an integer from 0 to 10;
n⁵ is an integer from 1 to 10;
R⁴, R⁵, R⁶, R⁷ are the same or different, and are H or straight or branched C₁-C₄-alkyl, preferably R⁴, R⁵, R⁶, R⁷ are H;
wherein the —ONO₂ group is linked to

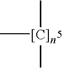

wherein n⁵ is as defined above;
Y² is an heterocyclic saturated, unsaturated or aromatic 5 or 6 members ring, containing one or more heteroatoms selected from nitrogen, oxygen, sulfur,
and is selected from

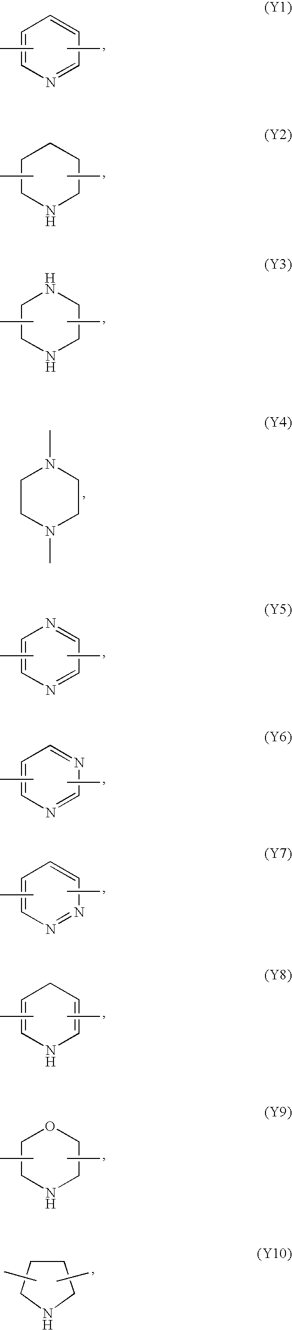

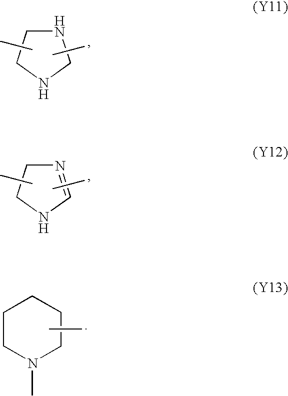

The following are preferred compounds according to the present invention:
fluvastatin 4-(nitrooxy)butyl ester,
fluvastatin 4-(nitrooxymethyl)benzyl ester,
fluvastatin 3-(nitrooxymethyl)benzyl ester,
fluvastatin 2-(nitrooxymethyl)benzyl ester,
fluvastatin 4-(nitrooxymethyl)phenyl ester,
fluvastatin 3-(nitrooxymethyl)phenyl ester,
fluvastatin 2-(nitrooxymethyl)phenyl ester,
fluvastatin 2-[2′-(nitrooxy)ethyloxy]ethyl ester,
fluvastatin 2-methoxy-4-[[4′-(nitrooxy)butyl]trans-2-propenoyloxy]phenol ester,
pravastatin 4-(nitrooxy)butyl ester,
pravastatin 4-(nitrooxymethyl)benzyl ester,
pravastatin 3-(nitrooxymethyl)benzyl ester,
pravastatin 2-(nitrooxymethyl)benzyl ester,
pravastatin 4-(nitrooxymethyl)phenyl ester,
pravastatin 3-(nitrooxymethyl)phenyl ester,
pravastatin 2-(nitrooxymethyl)phenyl ester,
pravastatin 2-[2′-(nitrooxy)ethyloxy]ethyl ester,
pravastatin 2-methoxy-4-[[4′-(nitrooxy)butyl]trans-2-propenoyloxy]phenol ester,
cerivastatin 4-(nitrooxy)butyl ester,
cerivastatin 4-(nitrooxymethyl)benzyl ester,
cerivastatin 3-(nitrooxymethyl)benzyl ester,
cerivastatin 2-(nitrooxymethyl)benzyl ester,
cerivastatin 4-(nitrooxymethyl)phenyl ester,
cerivastatin 3-(nitrooxymethyl)phenyl ester,
cerivastatin 2-(nitrooxymethyl)phenyl ester,
cerivastatin 2-[2′-(nitrooxy)ethyloxy]ethyl ester,
cerivastatin 2-methoxy-4-[[4′-(nitrooxy)butyl]trans-2-propenoyloxy]phenol ester,
atorvastatin 4-(nitrooxy)butyl ester,
atorvastatin 4-(nitrooxymethyl)benzyl ester,
atorvastatin 3-(nitrooxymethyl)benzyl ester,
atorvastatin 2-(nitrooxymethyl)benzyl ester,
atorvastatin 4-(nitrooxymethyl)phenyl ester,
atorvastatin 3-(nitrooxymethyl)phenyl ester,
atorvastatin 2-(nitrooxymethyl)phenyl ester,
atorvastatin 2-[2′-(nitrooxy)ethyloxy]ethyl ester,
atorvastatin 2-methoxy-4-[[4′-(nitrooxy)butyl]trans-2-propenoyloxy]phenol ester,
rosuvastatin 4-(nitrooxy)butyl ester,
rosuvastatin 4-(nitrooxymethyl)benzyl ester,
rosuvastatin 3-(nitrooxymethyl)benzyl ester,
rosuvastatin 2-(nitrooxymethyl)benzyl ester,
rosuvastatin 4-(nitrooxymethyl)phenyl ester,
rosuvastatin 3-(nitrooxymethyl)phenyl ester,
rosuvastatin 2-(nitrooxymethyl)phenyl ester,
rosuvastatin 2-[2′-(nitrooxy)ethyloxy]ethyl ester, and
rosuvastatin 2-methoxy-4-[[4′-(nitrooxy)butyl]trans-2-propenoyloxy]phenol ester.

The general synthesis of the NO-releasing statins of formula (I) is described in the WO 2004/105754.

### Effects of NO-Releasing Pravastatin in Human PASMCs

### Effects on DNA Synthesis

DNA synthesis was measured by [³H-methyl]-thymidine incorporation over 24 hours as previously described.

Human pulmonary artery smooth muscle cells (PASMCs) were seeded in 48-well plates at a density of 5×10⁴ cells/well in Dulbecc's modified Eagle medium (DMEM) containing 10% fetal bovine medium (FBS). Cells were allowed to adhere overnight, providing a monolayer of approximately 80-90% confluence, and then quiesced for 48 hours with daily changes in serum-free DMEM. Cells were subsequently incubated in fresh medium containing 0.25 μCi/well [³H-methyl]-thymidine (GE Healthcare, Little Chalfont, Buck's, UK) and treated with pravastatin and NO-releasing pravastatin (Example 1 of WO 2004/105754) at stated concentrations in the presence and absence of recombinant human platelet-derived growth factor-BB (PDGF) (5 ng/ml). Four to six replicates were analysed per treatment and [³H-methyl]-thymidine uptake determined by liquid scintillation analysis (2200CA TRI-CARB, United Technologies Packcard, Pangbourne, UK).

As shown in Table 1, differently from pravastatin, the NO-releasing derivative inhibits DNA synthesis.

TABLE 1

| | [³H-Methyl]-thymidine | | |
|---|---|---|---|
| | incorporation (% of PDGF) | | |
| Concentration | 1 μM | 5 μM | 10 μM |
| Pravastatin | 95.0 ± 3.6 | 92.5 ± 4.2 | 89.5 ± 2.4 |
| NO-releasing Pravastatin | 87.5 ± 9.8 | 55.3 ± 3.4 | 39.0 ± 3.0 |

### Effects on TGF-β1-Stimulated ET-1 Release

Endothelin-1 (ET-1) release from PASMCs was measured in conditioned media using a chemiluminescent immunoassay system (QuantiGlo®, R&D Systems, UK). Cells were grown to confluence in 24-well plates (5×10⁴ cells per well) in DMEM containing 10% FBS and serum-deprived for 24-hours prior to stimulation with transforming growth factor-31 (TGF-β1) (10 ng/ml) and treatment with pravastatin and NO-releasing pravastatin (Example 1 of WO 2004/105754). Release experiments were also conducted in the presence of mevalonic acid (MVA), farnesylpyrophosphate (FPP), geranylgeranylpyrophosphate (GGPP) and inhibitors of geranylgeranyl transferase (GGTI-2133), farnesyl transferase (FTI-277) and Rho Kinase (Y27632). Production of MMP-9 was induced by dual stimulation with tumour necrosis factor-α (TNF-α) (10 ng/ml) and phorbol 12-myrisate 13-acetate (PMA) (0.1 μM) and measured in conditioned medium using a Biotrack® ELISA-based immunoassay (GE Healthcare, UK).

In the presence of TGF-β1 (10 ng/ml), ET-1 production by PASMCs increased markedly, compared with control serum-deprived cells. The results reported in Table 2 show that the compound of the invention is effective in inhibiting ET-1 release. Conversely, the pravastatin had not effect.

TABLE 2

| | ET-1 |
|---|---|
| | (% of TGF-β1) |
| Pravastatin (1 μM) | 89.9 ± 12.8 |
| NO-releasing Pravastatin (1 μM) | 21.3 ± 6.6 |

1. Use of a nitric oxide-releasing statin of formula (I) 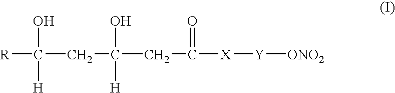
or pharmaceutically acceptable salts or stereoisomers thereof for the preparation of medicaments for the treatment of pulmonary arterial hypertension, wherein in the general formula (I),
R is 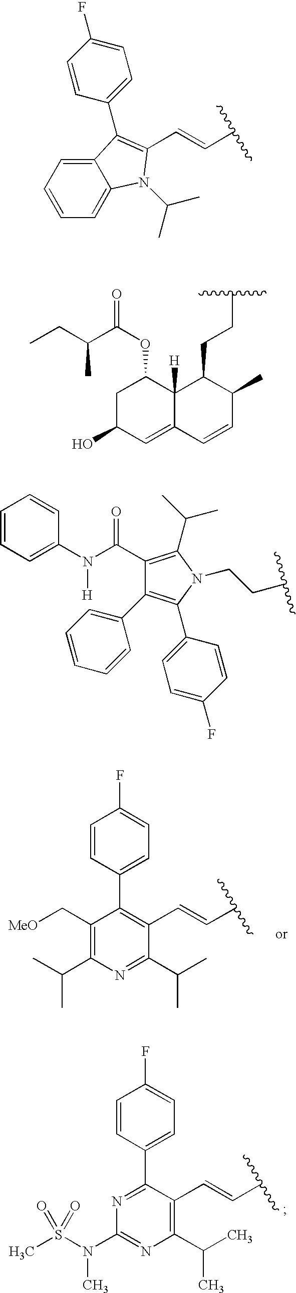
X is —O—, —S—, —NH— or —NHR′—, R′ being straight or branched alkyl with 1 to 10 carbon atoms, preferably CH₃;
Y is a bivalent radical having the following meaning:
a)
straight or branched C₁-C₂₀-alkylene, preferably having from 2 to 5 carbon atoms;
cycloalkylene with 5 to 7 carbon atoms into cycloalkylene ring, the ring being eventually substituted with side chains T, wherein T is straight or branched alkyl with from 1 to 10 carbon atoms, preferably CH₃; 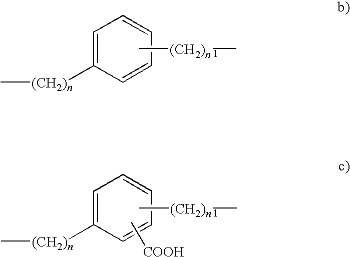
wherein n is an integer from 0 to 20, and n¹ is an integer from 1 to 20; 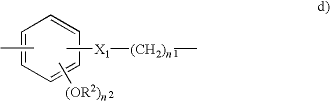
wherein:
n¹ is as defined above and n² is an integer from 0 to 2;
X₁=—OCO— or —COO— and R² is H or CH₃; 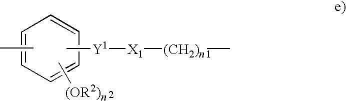
wherein:
n¹, n², R² and X₁ are as defined above;
Y¹ is —CH₂—CH₂— or (CH₂)ₙ²—; 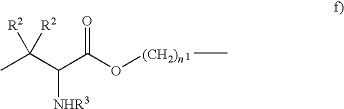
wherein:
n¹ and R² are as defined above, R³ is H or COCH₃;
with the proviso that when Y is selected from the bivalent radicals mentioned under b)-f), the —ONO₂ group is linked to a —CH₂ group; 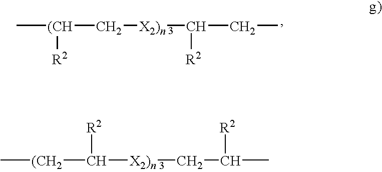
wherein X₂ is —O— or —S—, n³ is an integer from 1 to 6, preferably from 1 to 4, R² is as defined above; 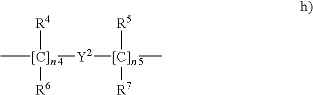
wherein:
n⁴ is an integer from 0 to 10;
n⁵ is an integer from 1 to 10;
R⁴, R⁵, R⁶, R⁷ are the same or different, and are H or straight or branched C₁-C₄-alkyl, preferably R⁴, R⁵, R⁶, R⁷ are H;
wherein the —ONO₂ group is linked to 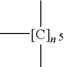
wherein n⁵ is as defined above;
Y² is an heterocyclic saturated, unsaturated or aromatic 5 or 6 members ring, containing one or more heteroatoms selected from nitrogen, oxygen, sulfur,
and is selected from

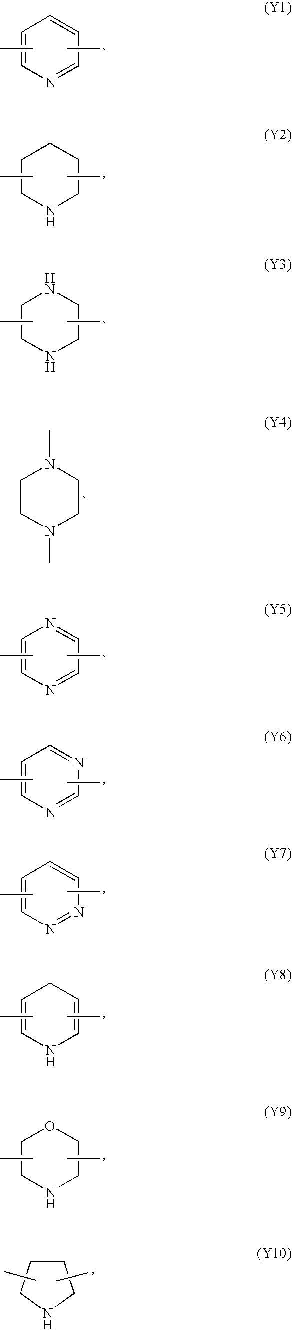

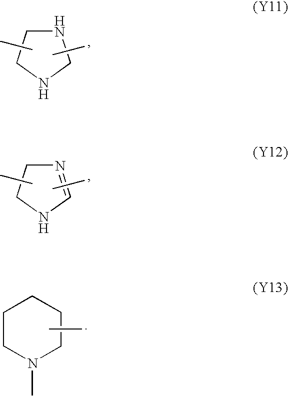

2. Use according to claim 1 wherein the compound of formula (I) is selected in the group consisting of:
fluvastatin 4-(nitrooxy)butyl ester,
fluvastatin 4-(nitrooxymethyl)benzyl ester,
fluvastatin 3-(nitrooxymethyl)benzyl ester,
fluvastatin 2-(nitrooxymethyl)benzyl ester,
fluvastatin 4-(nitrooxymethyl)phenyl ester,
fluvastatin 3-(nitrooxymethyl)phenyl ester,
fluvastatin 2-(nitrooxymethyl)phenyl ester,
fluvastatin 2-[2′-(nitrooxy)ethyloxy]ethyl ester,
fluvastatin 2-methoxy-4-[([4′-(nitrooxy)butyl]trans-2-propenoyloxy]phenol ester,
pravastatin 4-(nitrooxy)butyl ester,
pravastatin 4-(nitrooxymethyl)benzyl ester,
pravastatin 3-(nitrooxymethyl)benzyl ester,
pravastatin 2-(nitrooxymethyl)benzyl ester,
pravastatin 4-(nitrooxymethyl)phenyl ester,
pravastatin 3-(nitrooxymethyl)phenyl ester,
pravastatin 2-(nitrooxymethyl)phenyl ester,
pravastatin 2-[2′-(nitrooxy)ethyloxy]ethyl ester,
pravastatin 2-methoxy-4-[[4′-(nitrooxy)butyl]trans-2-propenoyloxy]phenol ester,
cerivastatin 4-(nitrooxy)butyl ester,
cerivastatin 4-(nitrooxymethyl)benzyl ester,
cerivastatin 3-(nitrooxymethyl)benzyl ester,
cerivastatin 2-(nitrooxymethyl)benzyl ester,
cerivastatin 4-(nitrooxymethyl)phenyl ester,
cerivastatin 3-(nitrooxymethyl)phenyl ester,
cerivastatin 2-(nitrooxymethyl)phenyl ester,
cerivastatin 2-[2′-(nitrooxy)ethyloxy]ethyl ester,
cerivastatin 2-methoxy-4-[[4′-(nitrooxy)butyl]trans-2-propenoyloxy]phenol ester,
atorvastatin 4-(nitrooxy)butyl ester,
atorvastatin 4-(nitrooxymethyl)benzyl ester,
atorvastatin 3-(nitrooxymethyl)benzyl ester,
atorvastatin 2-(nitrooxymethyl)benzyl ester,
atorvastatin 4-(nitrooxymethyl)phenyl ester,
atorvastatin 3-(nitrooxymethyl)phenyl ester,
atorvastatin 2-(nitrooxymethyl)phenyl ester,
atorvastatin 2-[2′-(nitrooxy)ethyloxy]ethyl ester,
atorvastatin 2-methoxy-4-[[4′-(nitrooxy)butyl]trans-2-propenoyloxy]phenol ester,
rosuvastatin 4-(nitrooxy)butyl ester,
rosuvastatin 4-(nitrooxymethyl)benzyl ester,
rosuvastatin 3-(nitrooxymethyl)benzyl ester,
rosuvastatin 2-(nitrooxymethyl)benzyl ester,
rosuvastatin 4-(nitrooxymethyl)phenyl ester,
rosuvastatin 3-(nitrooxymethyl)phenyl ester,
rosuvastatin 2-(nitrooxymethyl)phenyl ester,
rosuvastatin 2-[2′-(nitrooxy)ethyloxy]ethyl ester, and
rosuvastatin 2-methoxy-4-[[4′-(nitrooxy)butyl]trans-2-propenoyloxy]phenol ester.

3. A nitric oxide-releasing statin of formula (I) 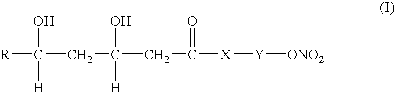
or pharmaceutically acceptable salts or stereoisomers thereof, wherein in the general formula (I),
R is 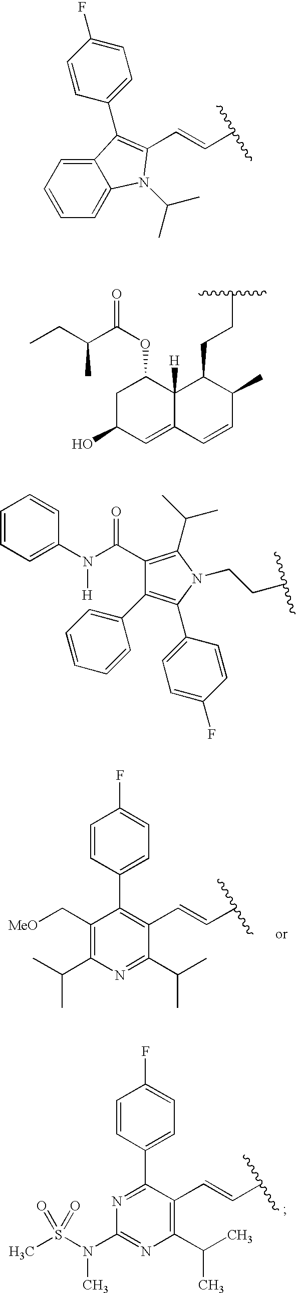
X is —O—, —S—, —NH— or —NHR′—, R′ being straight or branched alkyl with 1 to 10 carbon atoms, preferably CH₃;
Y is a bivalent radical having the following meaning:
a)
straight or branched C₁-C₂₀-alkylene, preferably having from 2 to 5 carbon atoms;
cycloalkylene with 5 to 7 carbon atoms into cycloalkylene ring, the ring being eventually substituted with side chains T, wherein T is straight or branched alkyl with from 1 to 10 carbon atoms, preferably CH₃; 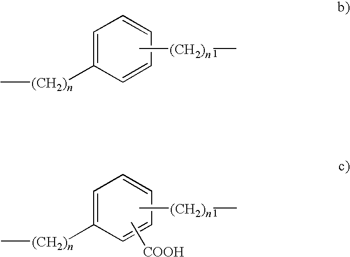
wherein n is an integer from 0 to 20, and n¹ is an integer from 1 to 20; 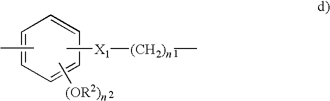
wherein:
n¹ is as defined above and n² is an integer from 0 to 2;
X₁=—OCO— or —COO— and R² is H or CH₃; 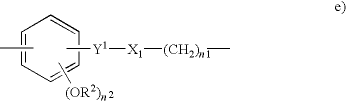
wherein:
n¹, n², R² and X₁ are as defined above;
Y¹ is —CH₂—CH₂— or —CH═CH—(CH₂)ₙ²—; 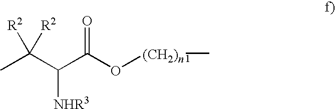
wherein:
n¹ and R² are as defined above, R³ is H or COCH₃;
with the proviso that when Y is selected from the bivalent radicals mentioned under b)-f), the —ONO₂ group is linked to a —CH₂ group; 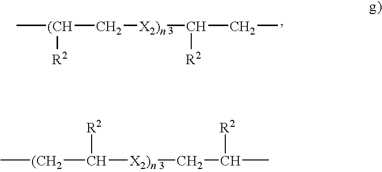
wherein X₂ is —O— or —S—, n³ is an integer from 1 to 6, preferably from 1 to 4, R² is as defined above; 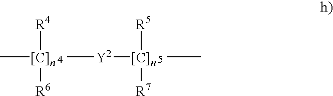
wherein:
n⁴ is an integer from 0 to 10;
n⁵ is an integer from 1 to 10;
R⁴, R⁵, R⁶, R⁷ are the same or different, and are H or straight or branched C₁-C₄-alkyl, preferably R⁴, R⁵, R⁶, R⁷ are H;
wherein the —ONO₂ group is linked to 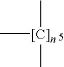
wherein n⁵ is as defined above;
Y² is an heterocyclic saturated, unsaturated or aromatic 5 or 6 members ring, containing one or more heteroatoms selected from nitrogen, oxygen, sulfur, and is selected from 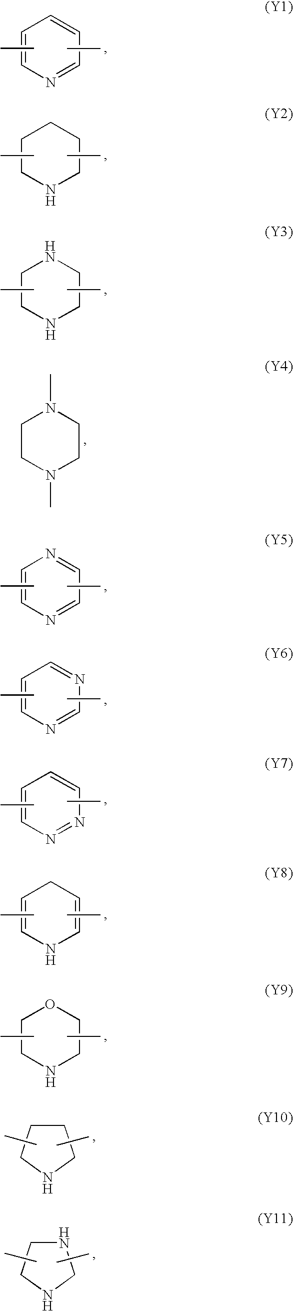 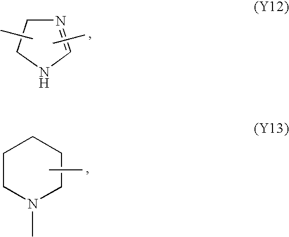
for use in the treatment of pulmonary arterial hypertension.

4. A compound of formula (I) selected in the group consisting of:
fluvastatin 4-(nitrooxy)butyl ester,
fluvastatin 4-(nitrooxymethyl)benzyl ester,
fluvastatin 3-(nitrooxymethyl)benzyl ester,
fluvastatin 2-(nitrooxymethyl)benzyl ester,
fluvastatin 4-(nitrooxymethyl)phenyl ester,
fluvastatin 3-(nitrooxymethyl)phenyl ester,
fluvastatin 2-(nitrooxymethyl)phenyl ester,
fluvastatin 2-[2′-(nitrooxy)ethyloxy]ethyl ester,
fluvastatin 2-methoxy-4-[[4′-(nitrooxy)butyl]trans-2-propenoyloxy]phenol ester,
pravastatin 4-(nitrooxy)butyl ester,
pravastatin 4-(nitrooxymethyl)benzyl ester,
pravastatin 3-(nitrooxymethyl)benzyl ester,
pravastatin 2-(nitrooxymethyl)benzyl ester,
pravastatin 4-(nitrooxymethyl)phenyl ester,
pravastatin 3-(nitrooxymethyl)phenyl ester,
pravastatin 2-(nitrooxymethyl)phenyl ester,
pravastatin 2-[2′-(nitrooxy)ethyloxy]ethyl ester,
pravastatin 2-methoxy-4-[[4′-(nitrooxy)butyl]trans-2-propenoyloxy]phenol ester,
cerivastatin 4-(nitrooxy)butyl ester,
cerivastatin 4-(nitrooxymethyl)benzyl ester,
cerivastatin 3-(nitrooxymethyl)benzyl ester,
cerivastatin 2-(nitrooxymethyl)benzyl ester,
cerivastatin 4-(nitrooxymethyl)phenyl ester,
cerivastatin 3-(nitrooxymethyl)phenyl ester,
cerivastatin 2-(nitrooxymethyl)phenyl ester,
cerivastatin 2-[2′-(nitrooxy)ethyloxy]ethyl ester,
cerivastatin 2-methoxy-4-[[4′-(nitrooxy)butyl]trans-2-propenoyloxy]phenol ester,
atorvastatin 4-(nitrooxy)butyl ester,
atorvastatin 4-(nitrooxymethyl)benzyl ester,
atorvastatin 3-(nitrooxymethyl)benzyl ester,
atorvastatin 2-(nitrooxymethyl)benzyl ester,
atorvastatin 4-(nitrooxymethyl)phenyl ester,
atorvastatin 3-(nitrooxymethyl)phenyl ester,
atorvastatin 2-(nitrooxymethyl)phenyl ester,
atorvastatin 2-[2′-(nitrooxy)ethyloxy]ethyl ester,
atorvastatin 2-methoxy-4-[[4′-(nitrooxy)butyl]trans-2-propenoyloxy]phenol ester,
rosuvastatin 4-(nitrooxy)butyl ester,
rosuvastatin 4-(nitrooxymethyl)benzyl ester,
rosuvastatin 3-(nitrooxymethyl)benzyl ester,
rosuvastatin 2-(nitrooxymethyl)benzyl ester,
rosuvastatin 4-(nitrooxymethyl)phenyl ester,
rosuvastatin 3-(nitrooxymethyl)phenyl ester,
rosuvastatin 2-(nitrooxymethyl)phenyl ester,
rosuvastatin 2-[2′-(nitrooxy)ethyloxy]ethyl ester, and
rosuvastatin 2-methoxy-4-[[4′-(nitrooxy)butyl]trans-2-propenoyloxy]phenol ester,
for use in the treatment of pulmonary arterial hypertension.
